(19) United States
(12) Patent Application Publication
(10) Pub. No.: US 2008/0132854 A1
(43) Pub. Date: Jun. 5, 2008

(54) Interchangeable retractable needle syringe with safety filling cannula

(75) Inventor: Fraser R. Sharp, Vancouver (CA)

Correspondence Address: NIXON & VANDERHYE, PC, 901 NORTH GLEBE ROAD, 11TH FLOOR, ARLINGTON, VA, 22203, US

(73) Assignee: Inviro Medical Devices, Inc., Lawrenceville, GA (US)

(21) Appl. No.: 11/979,360

(22) Filed: Nov. 1, 2007

Related U.S. Application Data
(60) Provisional application No. 60/856,036, filed on Nov. 2, 2006.

(51) Int. Cl. A61M 5/31

(52) U.S. Cl. 604/240

(57) ABSTRACT

A cannula and syringe assembly includes a syringe barrel having an opening at one end thereof defined by a forward edge; a needle carrier seated within the opening and attached to the syringe barrel; and a cannula attached to the needle carrier; wherein the cannula is provided with at least one engagement surface that engages the forward edge of the opening in the syringe barrel such that forces exerted on the cannula during use are transferred to the syringe barrel.

This application claims priority from Provisional Application No. 60/856,036, filed Nov. 2, 2006 (Attorney Dkt. No. 968-297), incorporated herein by reference.

### FIELD AND BACKGROUND OF THE INVENTION

Because of the large number of infectious diseases which can be spread by accidental needlestick injuries, a number of safer medical devices have been developed including retractable needle syringes. The retractable needles may be integral to the syringe and non-interchangeable. Alternatively, the needles may be connected to the syringe via a retractable needle carrier which is releasably connected to the front of the syringe so that it can be withdrawn into the barrel to prevent re use and accidental needlestick injury. The releasable needle carrier contain a standard Luer male connector to allow interface with standard Luer fitting female sharp metal needles or with a Luer fitting female semi-sharp cannula which can be used to fill the syringe. After filling the syringe with a semi-sharp “filling” cannula, the cannula can then be removed to allow attachment of a sharp metal hypodermic needle for injection. Alternatively, the Luer male connector can be used to directly connect to some other device or port such as a Luer activated valve.

To enable filling of the syringe it may be necessary to penetrate the stopper on a medication vial for example. When vial stopper penetration is carried out using a sharp metal needle, the forces required are relatively small and when transmitted to the retractable needle carrier of the syringe, are unlikely to disrupt the needle carrier barrel connection or cause premature retraction of the needle carrier. In fact, the watertight seal and connection between needle carrier and syringe is usually able to withstand the forces applied to it through the sharp metal needle when a vial stopper is punctured.

However, semi-sharp plastic or other cannula requires greater forces to effect penetration of a vial stopper. If these forces are applied directly to the needle carrier, there is a possibility that the needle carrier will be inadvertently disengaged from the front of the barrel of the syringe and pushed into the barrel. This will result in catastrophic malfunction and will prevent filling of the syringe and subsequent use thereof.

If the strength of the needle carrier barrel interface seal is by design or dimensional modification increased significantly to resist the inadvertent disengagement during vial stopper puncture, then the force required to disengage the needle carrier from the end of the barrel, as would normally occur during disabling and disposal, will also be significantly increased. It is likely that the increased force required would make the normal disabling process of disengagement of needle carrier from barrel and retraction of the needle carrier and sharp hypodermic needle ergonomically very difficult or even impossible.

If the syringe barrel needle carrier watertight seal interface is not required to resist the force generated during vial stopper penetration, the syringe disabling and needle retraction forces can remain relatively low and the disabling function will be within a range easily tolerated by the end user.

### SUMMARY OF THE INVENTION

In the exemplary and non-limiting embodiments disclosed herein, a method is provided for protecting the fluid and air seal between the needle carrier/internal syringe barrel surface when relatively strong forces are applied to the semi-sharp cannula or needle during vial stopper penetration. This is effected by transferring forces which could be destructive to this seal directly to the syringe barrel, rather than to the needle carrier. This is accomplished in the described embodiments by specific engineered and dimensional features of the needle or safety cannula and the syringe that diminish or eliminate the possibility of inadvertent dislodging of the needle carrier from the front of the barrel during the penetration of the vial stopper.

To accomplish this force transference, the following design aspects are taken into consideration:
a. The relative positions and dimensions of the needle or cannula, the needle carrier and the open end of the syringe barrel; and
b. The position and nature of the particular force transferal features, e.g., wings or lugs or other mechanisms such as disks or collars on the needle or cannula.

In the exemplary embodiments described herein, the force transferring mechanism is accomplished by an axial interaction, i.e., by an axial engagement between the wings or lugs (or other suitable structure) on the cannula and the open circumferential end of the syringe barrel. This configuration is advantageous in that additional functionality is achieved, i.e., the wings or lugs also facilitate simplified removal or attachment of the needle or cannula from vial or port stoppers, or the needle carrier on the syringe barrel. To transfer the forces effectively, it may be advisable in other situations to use a transfer mechanism with maximal contact with the end of the syringe barrel, such as a circular disk or collar having a diameter equal to or greater than the external diameter of the end of the syringe barrel (a variant ovoid, rectangular or some other shape of disk could also be utilized).

Accordingly, in one aspect, the present invention relates to a cannula and syringe assembly comprising: a syringe barrel having an opening at one end thereof defined by a forward edge; a needle carrier allowing interchangeability of cannulae or needles or other devices seated within the opening and attached to the syringe barrel; and a cannula attached to the needle carrier; wherein the cannula is provided with at least one engagement surface that engages the forward edge of the opening in the syringe barrel such that forces exerted on the cannula during use are transferred to the syringe barrel.

In another aspect, the invention relates to a cannula and syringe assembly comprising: a syringe barrel having an opening at one end thereof defined by a forward edge; a needle carrier seated within the opening and attached to the syringe barrel; a cannula attached to the needle carrier; wherein the cannula is provided with at least one engagement surface that engages the forward edge of the opening in the syringe barrel such that forces exerted on the cannula during use are transferred to the syringe barrel; wherein the at least one engagement surface comprises a pair of diametrically opposed wing members; and wherein the needle carrier is formed with an upper edge spaced at least axially inwardly of the forward edge of the syringe barrel opening.

In still another aspect, the invention relates to a cannula and syringe assembly comprising: a syringe barrel having an opening at one end thereof defined by a forward edge; a needle carrier seated within the opening and attached to the syringe barrel; a cannula attached to the needle carrier; wherein the cannula is provided with at least one engagement surface that engages the forward edge of the opening in the syringe barrel such that forces exerted on the cannula during use are transferred to the syringe barrel; wherein the at least one engagement surface comprises an annular collar; and wherein the needle carrier is formed with an upper edge spaced at least axially inwardly of the forward edge of the syringe barrel opening.

The disclosed embodiments will now be described in connection with the drawings identified below.

### BRIEF DESCRIPTION OF THE DRAWINGS

FIG. 1 is a side elevation, partially in section, of a safety filling cannula attached to a syringe barrel in accordance with one exemplary embodiment of the invention;

FIG. 2 (FIGS. 2A-D) illustrate various views of a safety cannula similar to that shown in FIG. 1 but with a Luer Lok® hub;

FIG. 3 (FIGS. 3A-3D) illustrate various views of a safety cannula similar to that shown in FIG. 2 but with a differently configured tip; and

FIG. 4 is similar to FIG. 1 but wherein the wings or lugs have been replaced by a collar.

### DETAILED DESCRIPTION OF PREFERRED EMBODIMENTS

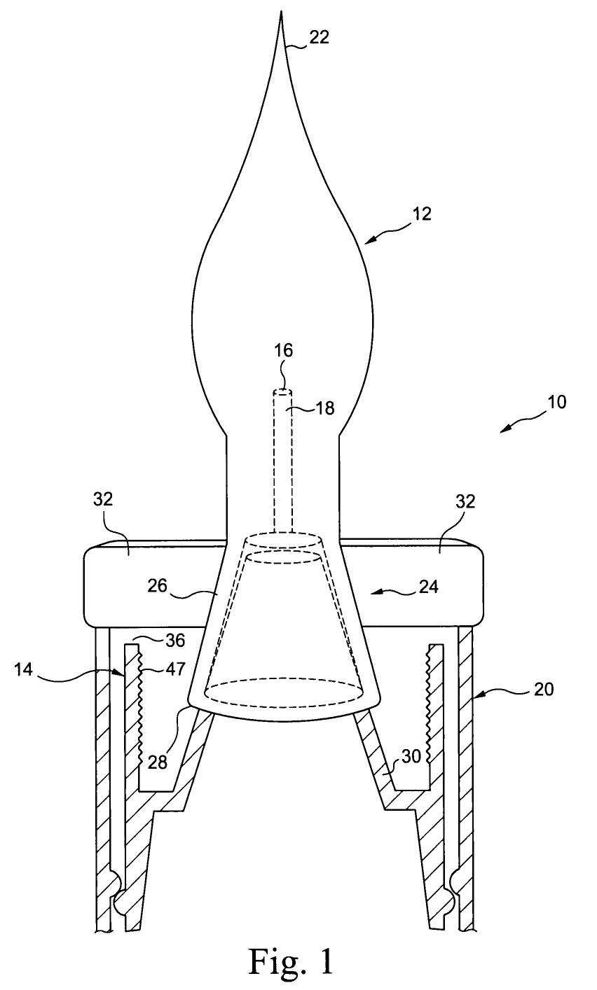

Referring to the drawings, particularly FIG. 1, a syringe generally designated 10 is illustrated including a solid-tip safety cannula 12 carried on a releasable needle carrier 14 with a lumen opening or port 16 through a side surface of the cannula in communication via an axial passage 18 with the interior of the syringe barrel 20. The tip 22 at the remote end of the cannula 12 is semi-sharp, enabling the cannula for penetration through the septum (i.e., an elastomeric membrane stopper of a vial, or a septum (possibly pre-slit) of an IV line access port). The relative bluntness of the tip 22 generally precludes penetration of the skin or of a protective glove as often worn by an individual using the syringe 10. The solid tip ensures that the insertion of the cannula through a membrane does not core the membrane or produce unwanted particles during insertion. The side opening port inevitably reduces the length of the fluid path compared to a passage which opens at the tip. In addition, since the passage does not traverse the narrow or narrowing portion of the cannula at its tip, relatively large diameter passages 16, 18 are permitted. These last two parameters reduce the resistance to fluid flow through the cannula, thus assisting filling and helping to reduce unwanted air entry and bubble formation within the syringe.

The cannula 12 connects to the needle carrier 14 by a Luer connection 24 (either a Luer fit or Luer Lok®). In this exemplary and non-limiting embodiment, the proximal end of the cannula 12 terminates in a hub portion 26 having a generally frusto-conically-shaped, hollow recess with a lower opening 28 for receiving in a friction-fit, a complimentary Luer-shaped and dimensioned fitting 30 on the end of the needle carrier 14 to thereby provide a conventional Luer fit connection. Finger flanges or wings 32 are provided adjacent the hub portion 26 of the cannula 12 to facilitate application and removal of the cannula relative to the needle carrier 14. Preferably, the finger flanges or wings 32 comprise a pair of lateral projections on the cannula body 12 circumferentially-spaced from one another by about 180°.

In accordance with the exemplary embodiment shown in FIG. 1, the finger flanges or wings 342 are sized and positioned, in concert with the size and shape of the forward end of the syringe barrel 20, and the male female Luer connection 30,26, so as to engage the upper edge 34 of the syringe barrel 20 upon attaching the cannula to the needle carrier 30. At the same time, the upper edge 36 of the needle carrier 14 is dimensioned to lie below the barrel edge 34 when the needle carrier is attached to the syringe barrel. This arrangement creates a gap 36 between the needle carrier and wings 32 so as to insure that any forces exerted on the cannula during insertion into a vial stopper, membrane or the like, will be transferred to the syringe barrel 20 and not to the needle carrier. Thus, the finger flanges or wings 32 not only serve to facilitate attachment and removal of the cannula from the needle carrier, but also serve as force transfer mechanisms that prevent accidental pushing of the cannula and needle holder into the syringe barrel.

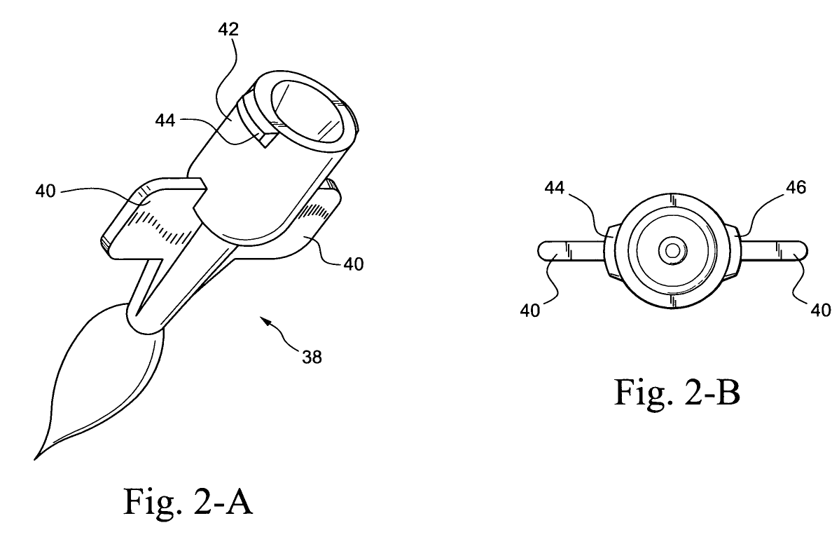

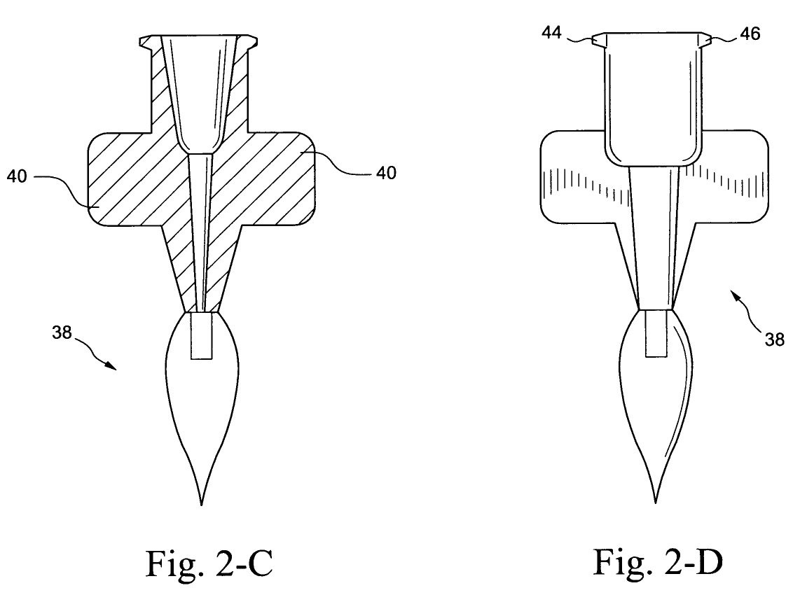

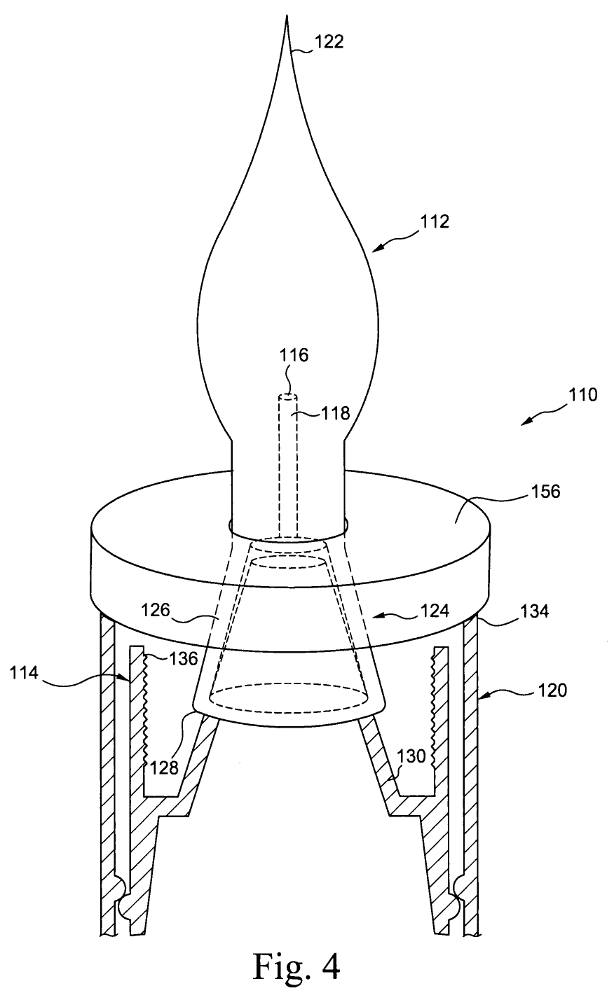

FIGS. 2-A through 2-D illustrate a blunt-tip cannula 38 in various views, the cannula having a pair of integral finger flanges (or force transfer wings) 40 in combination with a Luer female fitting 42 that cooperates with a mating Luer male hub on a needle carrier. In this regard, note the diametrically opposed tabs 44, 46 that are adapted to engage with complimentary threads 47 on the needle carrier (see FIGS. 1 and 4) in a conventional Luer Lok® arrangement.

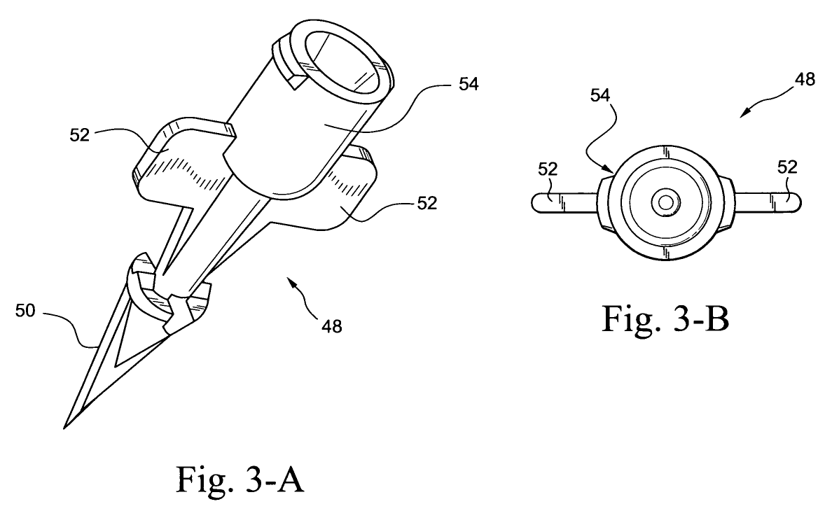

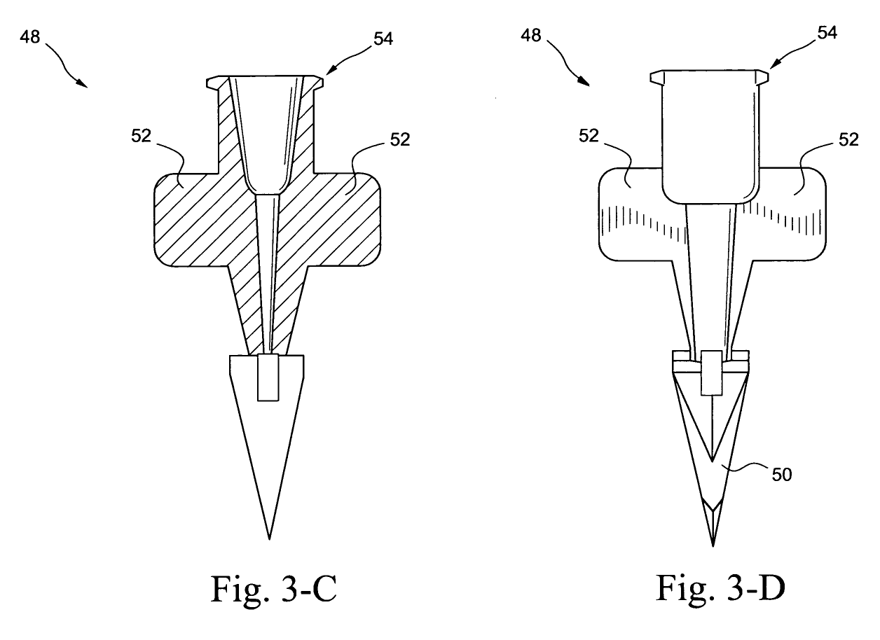

FIGS. 3-A through 3-D illustrate another blunt-tip cannula 48 having a differently configured distal tip 50, but also incorporating finger flanges (or force transfer wings) 52 in combination with a conventional Luer Lok® fitting 54. The manner in which wings 40, 52 in FIGS. 2A-2F and 3A-3F, respectively, operate to transfer forces from the cannula to the syringe barrel remains as described in connection with FIG. 1.

It will be appreciated that other mechanical mechanisms could be utilized to transfer force from the cannula to the syringe barrel, for example, a flat disc or collar could be substituted for the oppositely-directed wings described above. In addition, the needle carrier could be connected to the syringe barrel by a threaded connection. For example, in FIG. 4, an arrangement similar to FIG. 1 is shown (similar reference numerals are used to designate corresponding components, but with the prefix “1” added), but where the wings 32 have been replaced by a flat disc or collar 54 that is located to engage the upper edge 134 of the syringe barrel 120. The disc or collar can be of any suitable shape (for example, round, ovoid, square, triangular or the like), so long as at least a portion thereof extends beyond the diameter of the forward end of the syringe barrel, preferably to engage the barrel edge at two or more circumferentially spaced locations, and more preferably, to engage the barrel edge continuously.

While the invention has been described in connection with what is presently considered to be the most practical and preferred embodiment, it is to be understood that the invention is not to be limited to the disclosed embodiment, but on the contrary, is intended to cover various modifications and equivalent arrangements.

What is claimed is:

1. A cannula and syringe assembly comprising:
a syringe barrel having an opening at one end thereof defined by a forward edge;
a needle carrier allowing interchangeability of cannulae or needles or other devices seated within said opening and attached to said syringe barrel; and
a cannula attached to said needle carrier; wherein said cannula is provided with at least one engagement surface that engages said forward edge of said opening in said syringe barrel such that forces exerted on said cannula during use are transferred to said syringe barrel.

2. The assembly of claim 1 wherein said at least one engagement surface comprises an annular collar.

3. The assembly of claim 1 wherein said at least one engagement surface comprises a pair of diametrically opposed wing members.

4. The assembly of claim 1 wherein said needle carrier is formed with an upper edge spaced radially and axially inwardly of said forward edge of said syringe barrel opening.

5. The assembly of claim 1 wherein said cannula or needle is attached to said needle carrier by a sliding friction fit between two conical surfaces.

6. The assembly of claim 1 wherein said cannula or needle is attached to said needle carrier by a threaded connection.

7. A cannula and syringe assembly comprising:
a syringe barrel having an opening at one end thereof defined by a forward edge;
a needle carrier seated within said opening and attached to said syringe barrel;
a cannula attached to said needle carrier; wherein said cannula is provided with at least one engagement surface that engages said forward edge of said opening in said syringe barrel such that forces exerted on said cannula during use are transferred to said syringe barrel;
wherein said at least one engagement surface comprises a pair of diametrically opposed wing members; and
wherein said needle carrier is formed with an upper edge spaced at least axially inwardly of said forward edge of said syringe barrel opening.

8. The assembly of claim 7 wherein said cannula is attached to said needle carrier by a sliding friction fit between two conical surfaces.

9. The assembly of claim 7 wherein said cannula is attached to said needle carrier by a threaded connection.

10. A cannula and syringe assembly comprising:
a syringe barrel having an opening at one end thereof defined by a forward edge;
a needle carrier seated within said opening and attached to said syringe barrel;
a cannula attached to said needle carrier; wherein said cannula is provided with at least one engagement surface that engages said forward edge of said opening in said syringe barrel such that forces exerted on said cannula during use are transferred to said syringe barrel;
wherein said at least one engagement surface comprises an annular collar; and
wherein said needle carrier is formed with an upper edge spaced at least axially inwardly of said forward edge of said syringe barrel opening.

11. The assembly of claim 10 wherein said cannula or needle is attached to said needle carrier by a sliding friction fit between two conical surfaces.

12. The assembly of claim 10 wherein said cannula or needle is attached to said needle carrier by a threaded connection.
